(19) United States
(12) Patent Application Publication
(10) Pub. No.: US 2007/0118134 A1
(43) Pub. Date: May 24, 2007

(54) Surgical Tools and method to facilitate spinal surgery

(76) Inventor: Jeffery Thramann, Longmont, CO (US)

Correspondence Address: HOLLAND & HART, LLP, P.O BOX 8749, DENVER, CO, 80201, US

(21) Appl. No.: 11/251,689

(22) Filed: Oct. 17, 2005

(51) Int. Cl. A61B 17/00

(52) U.S. Cl. 606/79

(57) ABSTRACT

A surgical method, tool, and system to facilitate spinal surgery includes locating an inferior pedicle and guiding a bone cutting device to the inferior pedicle. The bone cutting device is used to remove a necessary portion of the vertebral body to provide access to the disc space. Typically the bone cutting device is removed and a speculum is inserted along the bone cutter guide until the speculum contacts a disc annulus between the inferior vertebral body and a superior vertebral body. The speculum is moved to ensure the nerves are out of the surgical area. The speculum also provides a shield to inhibit inadvertent damage to the nerve why the surgeon is operating.

### FIELD OF THE INVENTION

The present invention relates to surgical tools and methods and, more particularly, surgical tools and methods to facilitate spinal surgeries.

### BACKGROUND OF THE INVENTION

Intervertebral discs can have many problems that require a surgeon to access the intervertebral disc or disc space. For example, in some instances, a surgeon may need to implant a graft to facilitate fusing adjacent vertebral bodies, such as, for example, during a TLIF procedure. To implant the graft, the surgeon needs access to the vertebral disc to remove all or part of the disc nucleus and, in some cases, the disc annulus (generically referred to as “disc material”). After removal of the disc material, the surgeon implants a graft to facilitate fusion between the superior and inferior disc. Alternatively, a surgeon may implant an artificial disc instead of fusing the vertebral bodies. Frequently, to provide adequate access, the surgeon removes a portion of the bone associated with the vertebral column, such as, for example, a portion of the lamina, facet, or the like.

As can be appreciated, the surgical site is relatively small for the procedure. Furthermore, the spinal cord and other nerves are located relatively close to the surgical area. To safely perform the surgery, a surgeon, for example, may take a significant portion of the surgical time locating the various nerves and orienting themselves in the surgical area.

Thus, it would be desirous to develop a surgical tool and method that facilitates the spinal surgical procedures.

### SUMMARY OF THE INVENTION

To attain the advantages and in accordance with the present invention, a method to facilitate surgical access to a vertebral disc is provided. The method begins by locating an inferior pedicle and guiding a bone cutting device to the inferior pedicle. The bone cutting device is used to remove a portion of the vertebral body to provide access to the disc space. Typically the bone cutting device is removed and a speculum is inserted along the bone cutter guide until the speculum contacts and/or pierces a disc annulus between the inferior vertebral body and a superior vertebral body. The speculum is moved to ensure the nerves are out of the surgical area. The speculum also provides a shield to inhibit inadvertent damage to the nerve why the surgeon is operating.

The present invention also provides a system to facilitate spinal surgery. The system comprises a bone cutter and a guide coupled to the bone cutter. The guide facilitates placement of the bone cutter on a portion of the inferior vertebral body to be removed to provide surgical access. The system further includes a speculum that is coupled to the guide. The speculum has a surface proximate an annulus of the intervertebral disc. The surface being movable to distract the nerve from the surgical access, such that the system facilitates removal of bone and isolation of nerves to provide surgical access and reduce time in surgery.

Moreover, the present invention provides a tool to facilitate the removal of bone. The bone removal tool facilitates the surgical procedures. The bone removal tool comprises a bone cutter and a bone cutter guide. The bone cutter guide couples to or is integrated with the bone cutter. A track coupled to the bone cutter such that the bone cutter is movable on extenders such that the bone cutter can be moved to contact a vertebral body.

The foregoing and other features, utilities and advantages of the invention will be apparent from the following more particular description of a preferred embodiment of the invention as illustrated in the accompanying drawings.

### BRIEF DESCRIPTION OF THE DRAWING

The accompanying drawings, which are incorporated in and constitute a part of this specification, illustrate embodiments of the present invention, and together with the description, serve to explain the principles thereof. Like items in the drawings are referred to using the same numerical reference.

FIG. 1 is a anterior, lateral perspective view of a spinal segment;

FIG. 2 is a posterior, lateral perspective view of a vertebrae;

FIG. 3 is a flowchart illustrative of the surgical methodology consistent with an embodiment of the present invention;

FIG. 4 is a view of conventional pedicle screw extensions;

FIG. 5 is a perspective view of a part consistent with an embodiment of the present invention; and

FIG. 6 is a perspective view of a speculum consistent with an embodiment of the present invention.

### DETAILED DESCRIPTION

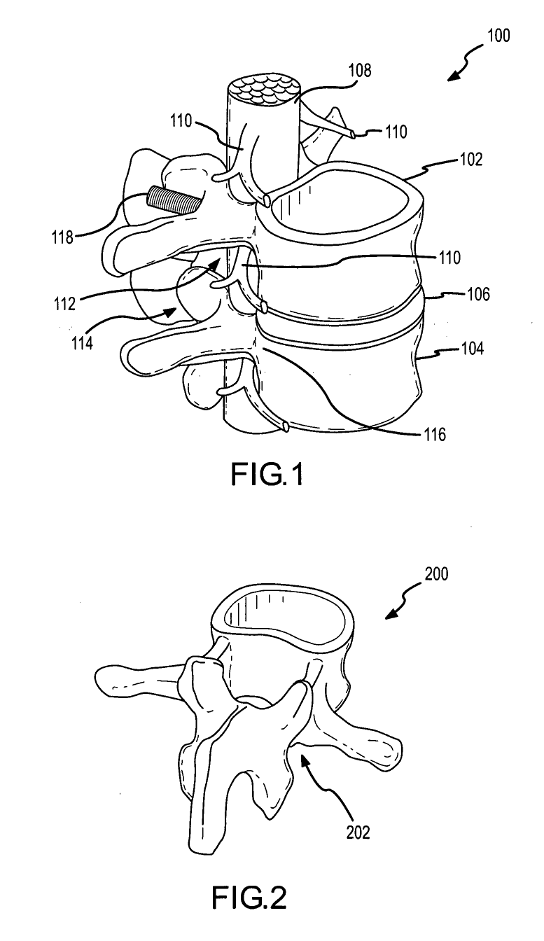

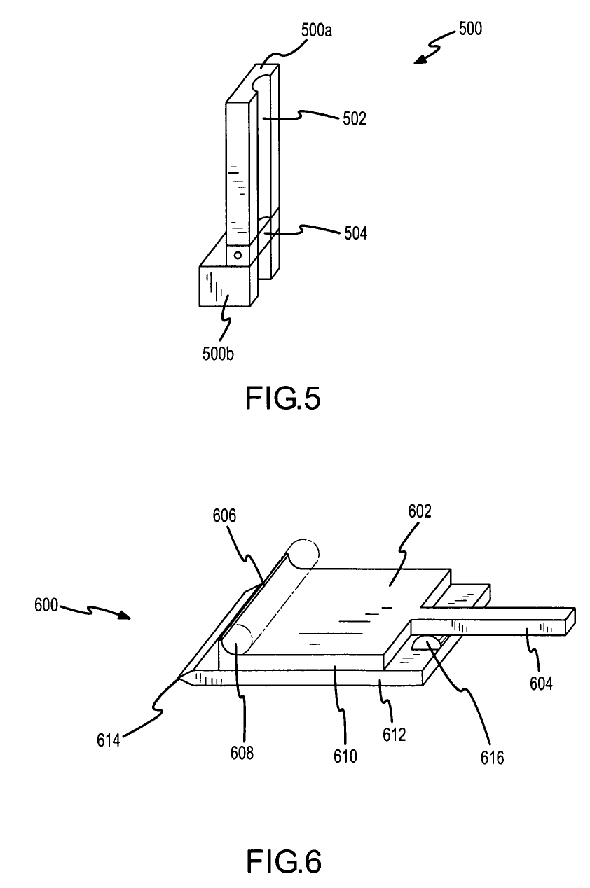

The present invention will now be described with reference to FIGS. 1 to 6. The drawings are provided for illustration and should not be considered limiting or to scale. Moreover, although the present invention is explained with specific reference to a TLIF procedure, one of ordinary skill in the art will recognize on reading the disclosure that the present invention may be used in other surgical procedures, such as, for example, a PLIF procedure, other fusion procedures, or the like.

FIG. 1 shows an anterior, lateral perspective view of a spinal segment 100. Segment 100 includes a superior vertebrae 102, an inferior vertebrae 104, and an intervertebral disc 106. A spinal cord 108 has a number of nerves 110 extending from the cord 108. As can be seen, the nerves 110 generally extend from the neural foramen 112 close to the pedicle of superior vertebrae 102.

In a TLIF procedure, for example, often the surgeon removes a portion of the inferior vertebrae 104, such as, for example, the facet 114, to allow surgical access to disc 106. FIG. 2 shows another view of segment 100 with a view of lamina 202 and facet 114. A rod may be implanted to facilitate fusion. In this case, a pedicle screw may be threaded into the vertebral body, such as pedicle screw 118 shown threaded into superior pedicle 102 in FIG. 1. Typically, two (2) screws 118 are used on each vertebral body although only one is shown for convenience. To facilitate fusion, rods are connected to screws 118 in a conventional manner, not shown or further described as it is generally well known in the art.

As can be appreciated, the surgeon performing the surgical procedure needs to take great care to avoid injury to spinal cord 108 and/or nerves 110. The present invention, however, provides orientation and safety features for the surgeon removing or reducing the time the surgeon needs to enter the disc space 106 and minimizing the potential injury to cord 108 and/or nerves 110. Moreover, the present invention provides a platform to allow the surgeon the ability to remove bone associated with the vertebral body and access the disc space in a reproducible and standardized fashion. Thus, the present invention reduces the overall surgical time and provides the ability for surgeons with less developed surgical skills to avoid damage to the patient and minimizes the learning curve thus leading to increased acceptance of, for example, minimally invasive fusion technology.

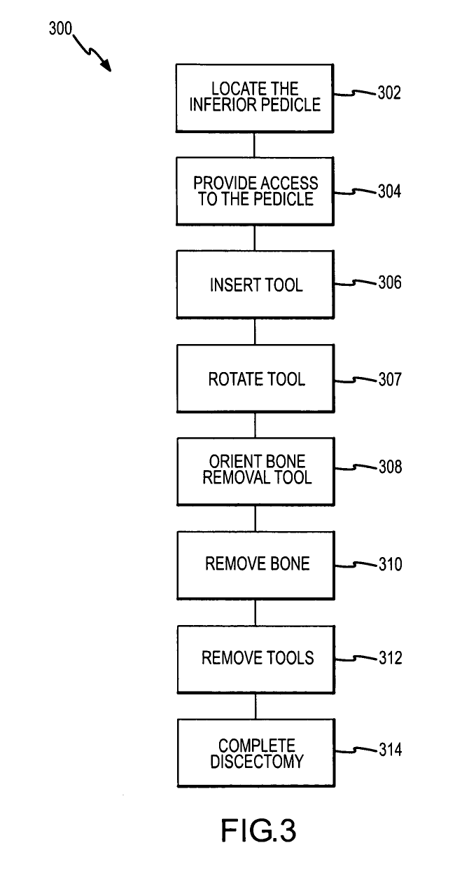

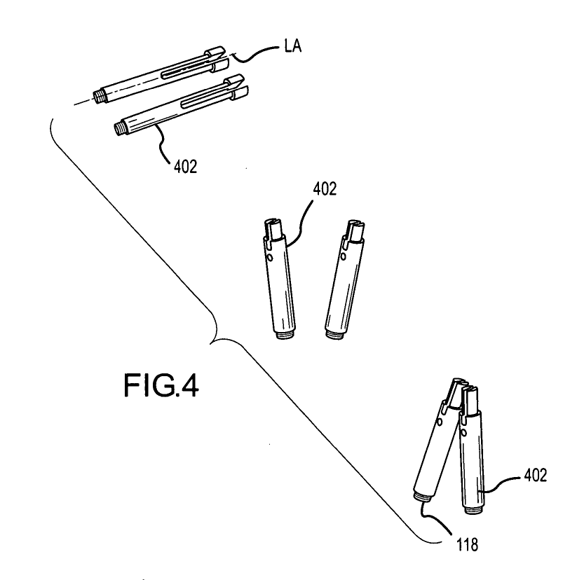

The present invention will be described with reference to FIG. 3, and flowchart 300 illustrating a use of the present invention. For convenience, flowchart 300 relates only to use of the present invention and does not address conventional surgical procedures, such as for a TLIF procedure. First, the surgeon would use conventional methods to cannulate the inferior pedicle, step 302. For example, the surgeon may use conventional guide wires and conventional surgical navigation technology to locate the inferior pedicle 116. Once the surgeon locates inferior pedicle 116, a dilator placed over the guide wire provides access to the pedicle, step 304. A pedicle screw 118 and extender 402 (see FIG. 4) as are generally known in the art may be threaded into pedicle 116. If a pedicle screw 118 and extender 402 are not used, an alternative structure may be placed that locates the pedicle and provides a track or stable platform.

After pedicle screw 118 and extender 402 or some other similar structure is provided, the surgeon would insert a bone cutter guide 500a and bone cutter 500b (collectively part 500), as shown bone cutter 500b is attached to bone cutter guide 500a as unitary member. Part 500 has a track or groove 502 that can clamp on, for example, extender 402. Part 500 movably couples to extender 402 or some similar structure. The movable connection could be slidable, geared, rotational, or the like. Part 500 would be moved along extender 402 using the track or groove 502 until cutter 500b abuts a portion of the vertebral body, step 306. In some instances, it is likely the tool will need to be rotated or angled (medially and laterally) about the long axis LA (FIG. 4) of the extender to properly align the bone cutter 500b. As shown, bone cutter 500b is a simple box shape. Other shapes are possible, such as, for example, a wedge shape, a circular shape, a conical shape, other random shapes, or the like.

Once placed next to the vertebral body, bone cutter 500b may need to be angularly oriented. Optionally, part 500 may have an angular orientation device 504 to align cutter 500b with the portion of the vertebral body to be removed. Angular orientation device 504 may be a simple cam or the like. If angular orientation is necessary, the device is oriented as necessary, step 308. Once oriented, the surgeon use the bone cutter 500b to remove portions of the vertebral body consistent with conventional surgical procedures, step 310, such as, for example, the surgeon may remove a portion of the facet joint. Bone cutter 500b may be shaped in a box shape as is conventional or other shapes, such as, circular, rectangular, triangular, other geometric or random shapes. Bone cutter 500b is used to remove a portion of the vertebral body only as necessary. If bone cutter 500b is hollow or has a through hole, bone cutter 500b may remain in place, but is preferably removed, step 312. A speculum 600 having a substantially flat portion 602 and handle 604 is inserted to the disc annulus, step 312. Flat portion 602 may terminate in a lip 606. Once placed speculum 600 is moved towards the superior pedicle. Flat portion 602 (and lip 606 is used) push on nerve 608 (shown in phantom, which corresponds to nerve 110) and holds nerve 608 against the superior pedicle 102. Underside 610 of speculum 600 provides a shield as the surgeon accesses disc 106. Once speculum 600 is placed such that nerve 608 is removed from the surgical area, the surgeon can complete the operation, 314. Optionally, speculum 600 may have a distractor 612, which may take many shapes by is generally flat and long similar to flat portion 602. Distractor 612 may extend beyond flat portion 602 and terminate in a leading edge 614. Leading edge may pierce the disc annulus to provide a platform or base for the surgical sight. Speculum 600 may be placed to maintain separation of vertebral bodies to facilitate access to disc 106. Distraction or separation of distractor 612 and flat portion 602 may be obtained by, for example, turning a cam 616, a scissor operation, or the like.

An embodiment of the present invention has been described with a degree of particularity. It should be understood that this description has been made by way of example, and that the invention is defined by the scope of the following

I claim:

1. A method to facilitate surgical access to a vertebral disc, the method comprising the steps of:
locating an inferior pedicle;
placing a bone cutter guide at the inferior pedicle;
guiding a bone cutter to the inferior pedicle using the bone cutter guide;
removing a portion of the inferior vertebral body using the bone cutter;
inserting a speculum along the bone cutter guide until the speculum contacts a disc annulus between the inferior vertebral body and a superior vertebral body; and
moving the speculum to provide access to the vertebral disc and protect the nerve during surgery, such that the surgeon can piece the disc annulus and access the disc nucleus.

2. The method of claim 1, wherein the step of locating the inferior pedicle comprises using surgical navigation technology.

3. The method of claim 1, further comprising the step of threading a pedicle screw into the inferior pedicle with a pedicle screw extender, and wherein the bone cutter is placed using the pedicle screw and pedicle screw extender as an orientation tool to place the bone cutter guide.

4. The method of claim 1, further comprising angularly orienting the bone cutter prior to removing the portion of the inferior vertebral body.

5. The method of claim 1, further comprising a distractor on the speculum to separate the inferior vertebral body and the superior vertebral body.

6. The method of claim 5, where in the distractor distracts the inferior vertebral body and the superior vertebral body when it is turned.

7. The method of claim 1, wherein the bone cutter is removed prior to insertion of the speculum.

8. The method of claim 1, wherein the step of inserting the speculum further comprises piercing the disc annulus.

9. A system to facilitate surgical access to an intervertebral disc between an inferior vertebral body and a superior vertebral body, the system comprising:
a bone cutter;
a guide coupled to the bone cutter, the guide to facilitate placement of the bone cutter on a portion of the inferior vertebral body to be removed to provide surgical access; and
a speculum coupled to the guide, the speculum have a flat surface proximate an annulus of the intervertebral disc, the flat surface being movable to distract the nerve from the surgical access, wherein
the system facilitates removal of bone and isolation of nerves to provide surgical access and reduce time in surgery.

10. The system of claim 9, wherein the guide comprises a track.

11. The system of claim 10, wherein the track couples to a pedicle screw extender.

12. The system of claim 9, wherein the speculum comprises a lip, the lip and flat surface cooperating to lift and shield the nerve.

13. The system of claim 9, wherein the speculum comprises a distractor.

14. The system of claim 13, wherein the distractor operates to distract the vertebral bodies by rotation.

15. The system of claim 13, further comprising an angular orientation device to orientate the bone cutter with the vertebral body.

16. The system of claim 13, wherein the bone cutter comprises a shape selected from the group of shapes consisting of: a box shape, a rectangular shape, a triangular shape, a circular shape, an elliptical shape, or a polygon shape.

17. A bone removal tool to facilitate surgical procedures, the tool comprising:
a bone cutter;
a bone cutter guide, the bone cutter guide coupled to the bone cutter; and
a track, the track coupled to the bone cutter such that the bone cutter is movable on extenders such that the bone cutter can be moved to contact a vertebral body.

18. The bone removal tool of claim 17, further comprising a speculum to engage, lift, and shield a nerve from a surgical area.

19. The bone removal tool of claim 18, further comprising an angular orientation device.

20. The bone removal tool of claim 18, further comprising a distractor having a leading edge to pierce a disc annulus.
